SEQUENCE LISTING (2 sequences)

SEQ ID NO: 1 - DNA, 768 bp, Artificial Sequence
  CDS (1)..(765): Tabacus yao Macquart
atgacttccataccggtgtccagctttttacttgcggccttagtactacaatacgccactagcgacgcagttaactactgtagattgccttgtcgaggggataattaccatgtgggctgtggtgagccagcgtatgcccaggaatgtgggcaaagtccaagaactcgcgagttattgaaggagcatagaaatgagattttgtcgaagatcaacgatgtgcgagaccatgtggccaagggatcatggggactaccagtggcagcccgaatgaaagttgttgtatgggatgcggaactcgctggtttagccaaacgacatacaaagggatgcgttggagagacacatgcgtgtcgaaacacagaacgtttctggttgccaggtcaactaaacttcaaatactccggcgacaagctaccccgcattaaggaacttattgatgatgctgtcaaaaaaggccacttgcagaagcataatattacgagagaaattatagggaactatagggagaatggacctaatggggatgtcaaggaacttgccttggccatatctgatcgagtcaccgccgttggctgcggactaactacttggcaggatggagcaaaggctcgcgcacttcttacgtgtaacttctcctcgcagaacactcggggtcgacctgtctacaaaatcggcaatagtcctggcgaaaagtgcatcgagaaggatgaaacgtataaaaacttgtgccctgctactgaacctatcgaccctaataagtctaactag

SEQ ID NO: 2 - protein, 255 aa, Artificial Sequence
  Tabacus yao Macquart
MTSIPVSSFLLAALVLQYATSDAVNYCRLPCRGDNYHVGCGEPAYAQECGQSPRTRELLKEHRNEILSKINDVRDHVAKGSWGLPVAARMKVVVWDAELAGLAKRHTKGCVGETHACRNTERFWLPGQLNFKYSGDKLPRIKELIDDAVKKGHLQKHNITREIIGNYRENGPNGDVKELALAISDRVTAVGCGLTTWQDGAKARALLTCNFSSQNTRGRPVYKIGNSPGEKCIEKDETYKNLCPATEPIDPNKSN

(19) United States
(12) Patent Application Publication
(10) Pub. No.: US 2012/0183525 A1
(43) Pub. Date: Jul. 19, 2012

(54) FIBRINOGENOLYTIC ENZYME TABFIBLYSIN OF HORSEFLY, TABANUS YAO, ENCODING GENE AND USE THEREOF

(76) Inventors: Ren Lai, Kunming (CN); Dongying Ma, Kunming (CN)

(21) Appl. No.: 13/381,685

(22) PCT Filed: Jun. 14, 2010
(86) PCT No.: PCT/CN2010/073987

§ 371 Date: Mar. 30, 2012

(30) Foreign Application Priority Data
Jul. 2, 2009 (CN) 200910094685.X

(51) Int. Cl. A61K 38/48; C07H 21/04; A61P 7/02; C12N 9/50

(52) U.S. Cl. 424/94.63; 435/219; 536/23.2

(57) ABSTRACT

A horsefly protease tabfiblysin isolated from the salivary gland of the horsefly, Tabanus Yao, a gene encoding the protease and use thereof are disclosed by the present invention. It belongs to the technical field of biomedicine. The molecular weight of the horsefly protease tabfiblysin is 27145.5 Daltons. Its full-length sequence is composed of 255 amino acids. Its encoding sequence is composed of 768 nucleotides. The horsefly protease tabfiblysin can hydrolyze fibrinogen and inhibit the aggregation of blood platelet dramatically, and can be used for preparing the drug for treating thrombotic diseases.

### TECHNICAL FIELD

This invention relates to a fibrinogenolytic enzyme tabfiblysin of horsefly, tabanus Yao, and its encoding gene and application. It belongs to the technical field of biomedicine.

### BACKGROUND OF THE INVENTION

Thrombotic diseases are a type of disease that seriously affects human health. In particular, cardiac and brain blood vessel thrombotic diseases have become the first cause of death in China. Timely use of anti-thrombus drugs can greatly increase patient persistence and reduce extent and scope of harm to viscera. Such anti-thrombus drugs mainly include those that dissolve thrombus, anticoagulant drugs, and anti-platelet drugs. Existing anticoagulant drugs and anti-platelet drugs have side effects of hemorrhage, thrombopenia, and cease of effect after stop of use of the drug. Therefore, people are actively looking for various new highly efficient, distinctive, and low toxicity anti-thrombus drugs from biological sources.

Gadfly family of insect is commonly referred to as horsefly. This insect belongs to arthropoda phylum diptera order brachycera suborder gadfly family. It is an important insect in medicine and theriatrics. Its adult is a traditional Chinese medicinal material commonly referred to as gadfly and features promotion of blood circulation to dispel blood stasis and breaking of dysmenorrheal. Use of this drug has a long history and it can provide drone and revelation for development of new modern drug. On the other hand, complexity of compositions of traditional Chinese medicine and method of processing of such medicine limit functioning of its active constituents. For this reason, locating specific active monomer in such traditional medicines is an important content of modernization of traditional Chinese medicines.

### SUMMARY OF THE INVENTION

This invention aims at provision of fibrinogenolytic enzyme of horsefly, tabanus Yao, and its encoding gene and application, based on existing technologies. Technical scheme of this invention is described below.

Tabanus Yao protease tabfiblysin is an active protein separated by us from salivary gland of tabanus Yao for the first time in the world. The molecular weight of this protease is 27145.5 Daltons. Its full-length sequence primary structure is the amino acid sequence shown by SEQ ID NO: 2.

Cloning of genes of tabanus Yao salivary gland protease tabfiblysin includes: extraction of total RNA of tabanus Yao salivary gland, purification of mRNA, mRNA reverse transcription, and construction of cDNA library. Primer is designed and PCR method is used to screen encoding gene. Results of gene sequencing show that tabfiblysin encoding sequence is composed of 768 nucleotides. Sequence from end 5′ to end 3′ is the nucleotide sequence shown be SEQ ID NO:1.

Tabanus Yao salivary gland protease tabfiblysin of this invention has apparent effect of hydrolysis of fibrinogen and suppression of platelet aggregation, and can be used as a drug to treat thrombotic diseases.

### DESCRIPTION OF DRAWING FIGURES

FIG. 1 shows Sephadex G-75 filtration chromatography of tabanus Yao salivary gland homogenate (SGE).

In this figure, the arrow points to active peak.

FIG. 2 shows SDS-PAGE analysis of fibrinogen hydrolysis effect of this invention.

FIG. 3 shows effect of tabfiblysin on human platelet aggregation induced by ADP.

### PREFERRED EMBODIMENTS

### Preferred Embodiment I

### Cloning of Gene of Tabanus Yao Salivary Gland Protease Tabfiblysin

I. Extraction of total RNA of tabanus Yao salivary gland:

A. Perform vivisection of tabanus Yao (from Shanxi Province of China; scientific name is T. yao Macquart). Pick out the salivary gland and instantly place it in liquid nitrogen. Quickly take certain amount (0.5-2 mg) of salivary gland tissue and add it into precooled Trizol extraction buffer solution (product of Invitrogen, USA). Place the mixture on ice for homogenate for 15 min.

B. Add chloroform of ⅕ the volume of Trizol and shake fiercely for 15 s. Place the mixture under room temperature for 5 min. Perform centrifugation at 4° C. and 12000 rpm for 10 min. Take supernatant.

C. Add the supernatant in iso-propyl alcohol of the same volume, and place the mixture under room temperature for 10 min. Perform centrifugation at 4° C. and 12000 rpm for 10 min. After precipitation, use 75% alcohol to wash once. Allow airing; precipitate at tube bottom is total RNA of tabanus Yao salivary gland.

II. Purification of tabanus Yao salivary gland mRNA:

Separation and purification of tabanus Yao salivary gland mRNA adopts PolyATtract® mRNA Isolation Systems kit of PROMEGA, USA.

A. Take 500 μg of tabanus Yao salivary gland total RNA and dissolve it in 500 μL DEPC. Place the solution in 65° C. water bath for 10 min. Then, add 3 μL of Oligo (dT) probe and 13 μL of 20×SSC solution. Mix even. Place the mixture under room temperature for cooling. The resultant solution is referred to as liquid A.

B. Washing of magnetic beads (SA-PMP): Lightly tap magnetic beads to mix them even. Move them to the magnetic rack for 30 s of adsorption. Discard supernatant. Add 300 μL of 0.5×SSC. Move the lot to the magnetic rack for 30 s of adsorption. Finally, add 100 μL of 0.5×SSC. The resultant liquid is referred to as liquid B.

C. Add liquid A into liquid B and place the mixture under room temperature for 10 min. Move this mixture to the magnetic rack for 30 s of adsorption. Discard supernatant. Wash 4 times using 0.1×SSC. Finally, discard supernatant. Add 100 μL of DEPC water suspension. Move the lot onto the magnetic rack for 30 s of adsorption. Transfer the supernatant to a new test tube and add 150 μL of DEPC water suspension. Move this to magnetic rack for 30 s of adsorption. Transfer supernatant to aforesaid test tube; this is purified tabanus Yao salivary gland mRNA.

D. Add 3M sodium acetate (pH5.2) of 1/10 volume and equal volume of iso-propyl alcohol. Place the lot at −70° C. for 30 min. Perform centrifugation at 4° C. and 12000 rpm for 10 min. Discard supernatant. Dissolve the precipitate in 10 μL of DEPC.

III. Construction of tabanus Yao salivary gland cDNA library: Creator™ SMART™ cDNA Library Construction Kit plasmid cDNA library construction kit of CLONTECH is adopted.

A. Synthesis of cDNA first chain (reverse transcription of mRNA):

1. In sterile PCR tube, add 1 μL of tabanus Yao salivary gland mRNA, 1 μL of SMART IV primer, 1 μL of CDS III/3′PCR primer, and 2 μL of deionized water, so that total volume reaches 5 μL.

2. Mix the lot even and perform short centrifugation. Place it at 72° C. for 2 min.

3. Place the centrifuge tube on ice for 2 min of incubation.

4. In the centrifuge tube, add the following reagents: 2.0 μL of 5× first chain buffer, 1.0 μL of 20 mMDTT, 1.0 μL of 10 mM dNTP mixture, and 1.0 μL of PowerScript reverse transcriptase.

5. Mix reagents in the centrifuge tube and perform short centrifugation. Place it at 42° C. for 1 hour.

6. Place the centrifuge tube on ice to interrupt synthesis of the first chain.

7. Take 2 μL of synthesized cDNA first chain from the centrifuge tube for later use.

B. Amplify to obtain second chain using the method of long terminal polymerase chain reaction (LD-PCR)

1. Preheat PCR instrument at 95° C.

2. Place 2 μL of cDNA first chain (mRNA reverse transcription), 80 μL of deionized water, 10 μL of 10×Advantage 2 PCR buffer, 2 μL of 50×dNTP mixture, 2 μL of 5′PCR primer, 2 μL of CDS III/3′ PCR primer, and 2 μL of bacillus coli polymerase in centrifuge tube for reaction.

3. In PCR instrument, perform amplification in the following sequence: 20 s at 95° C.; 22 cycles: 5 s at 95° C. followed by 6 min at 68° C.

4. After the cycles, extract synthesized cDNA dual chain in the centrifuge tube.

C. Preparation of bacillus coli DH5α competent cells:

1. Select single DH5α colony (purchased from Beijing Tiangeng Biochemical Science & Technology Co. Ltd.) and inoculate it to 3 mL of LB culture medium that does not contain ampicillin. Culture overnight at 37° C. In the next day, take aforesaid bacterium liquid and inoculate it to 50 mL of LB culture medium in proportion of 1:100. Oscillate for 2 hours at 37° C. When OD₆₀₀ value reaches 0.35, harvest bacterial culture. Place it in ice bath for 10 min. Centrifuge it at 4° C. and 5000 rpm for 10 min, to recover cells.

2. Add 600 μL of pre-cooled 0.1 mol/LCaCl₂-MgCl₂ solution heavy suspension in each mL of initial culture liquid.

3. Carry out centrifugation at 4° C. and 5000 rpm for 10 min to recover cells.

4. Add 60 μL of pre-cooled 0.1 mol/L CaCl₂ heavy suspension cell precipitate in each mL of initial culture, and sub-pack resultant liquid for later use.

D. Enzyme cutting, connection, and transformation of connection product:

1. In micro-centrifugal tube, add 1 μL of pMD19-T carrier (purchased from Takara, Japan) and 4 μL of tabanus Yao salivary gland cDNA dual chain solution. Full dose is 5 μL.

2. Add 5 μL (tales dose) of ligase buffer mixture.

3. Allow reaction for 2 hours at 16° C.

4. Add full dose (10 μL) to 100 μL of DH5α competent cells and place the lot in ice bath for 30 min.

5. After heating at 42° C. for 90 s, place it again in ice for 1 min.

6. Add 900 μL of LB culture medium that had been bathed at 37° C.; slowly shake it at 37° C. for 60 min of cultivation.

7. Take 200 μL of the above and apply it on LB culture medium containing X-Gal, IPTG, and Amp at 37° C. for 16 hours of cultivation, to form single colony.

8. In each LB flat dish, use 5 mL of LB liquid culture medium to wash colony. Add 30% glycerin for frozen storage. cDNA constructed contains about 1×10⁶ separate clones.

IV. Tabanus Yao salivary gland protease tabfiblysin gene order measurement and results:

According to inner peptide amino acid sequence of tabanus Yao salivary gland protease tabfiblysin obtained by separation and purification, and preference of dipster codon, we have designed a degenerate primer NT3-F21, to match joint primer CDSIII used for construction of tabanus Yao salivary gland cDNA library. Positive primer sequence and reverse primer sequence are:

NT3-F21:
5′-Tac tcc gg(g/c) gac aa(a/g) ct(g/a) cc(c/g)
cgc-3′;
CDSIII primer:
5′-att cta gag gcc gag gcg gcc atg-3′

In which two basic groups in parentheses indicate degenerate primer.

Take NT3-F21 and 3′PCR as primer, in constructed tabanus Yao salivary gland cDNA library, screen clone of cDNA sequence in which code tabfiblysin is inserted.

Reverse primer is designed according to measured nucleotide sequence of mature protein, and 5′ end conservative sequence cloned by cDNA library constructed is used as positive primer. Positive primer sequence and reverse primer sequence are:

5′primer:
5′-aag cag tgg tat caa cgc aga gt-3′
NT3-R21:
5′-(c/g)gg (t/c)ag (c/t)tt gtc (g/c)cc gga
gta-3′

Perform PCR with tabanus Yao salivary gland cDNA constructed as template. Reaction conditions: pre-denaturation at 95° C. for 4 min, followed by 35 cycles at the following conditions: 30 s at 94° C., 30 s at 58° C., and 40 s at 72° C. Next, place at 72° C. for 10 min. Using this method, in tabanus Yao salivary gland cDNA library constructed, screen cDNA sequence in which code Tabfiblysin is inserted. Measurement results show that cDNA sequence of code tabfiblysin is composed of 768 nucleotides. Sequence from 5′ end to 3′ end is the nucleotide sequence shown by SEQ ID NO:1.

### Preferred Embodiment II

### Preparation of Tabanus Yao Salivary Gland Protease Tabfiblysin

I. Tabanus Yao salivary gland dissection, homogenate, and treatment

Method of dissection of tabanus Yao (from Shanxi Province of China; scientific name is T. yao Macquart): Use small scissors to cut open back of the insect along its centerline. Soak it in 4° C. 0.01M phosphate buffer (pH7.2) containing 0.15M NaCl. Pick out the salivary gland and place it in a beaker containing aforesaid buffer solution (at −20° C.). After dissection, carry out centrifugation at 10000 rpm for 10 min. Continue to store the supernatant at −20° C. After thorough homogenate in precipitation glass homogenizer on ice, carry out centrifugation at 10000 rpm for 10 min. Combine supernatants from these two centrifugation operations, carry out freeze drying, and take a sample for dissolution in water, desalination by Sephadex G-25, and freeze drying.

II. For each step of separation and purification, activity is followed up using fibrinogen hydrolysis method.

Step 1: Sephadex G-75 gel filtration: Dissolve tabanus Yao salivary gland homogenate freeze-dried powder in 0.05M Tris-HCl+0.1M NaCl buffer solution (pH7.2). Apply a sample of this solution to Sephadex -75 (product of Pharmacia) solvent resistant column (1000 mm long; dia. of 26 mm) Use the same solution for elution and collect active constituents.

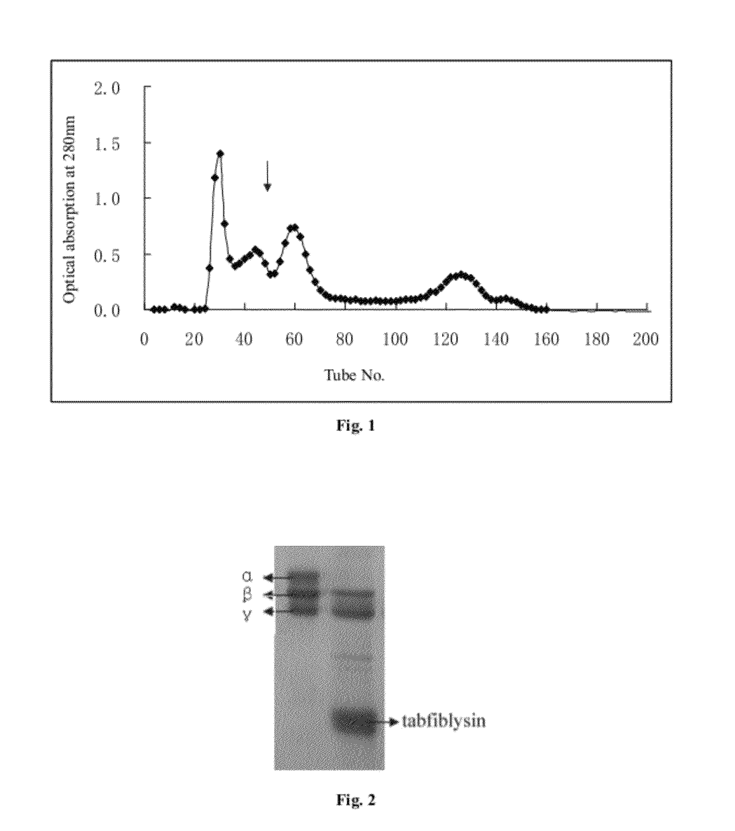

Step 2: FPLC Resource S ion exchange chromatography: Freeze dry active constituent peak (FIG. 1) obtained by Sephadex G-75 gel filtration and dissolve it in water. Carry out dialysis of the same in 0.05M Tris-HCl pH8.3 buffer solution for 12 hr. Apply a sample thereof to Resource S column, and use 0-0.5M NaCl linear gradient for elution. Purification yields fibrinogen hydrolytic enzyme. This step is performed in AKTA Purifier FPLC system.

### Preferred Embodiment III

### Pharmacological Experiments of Tabanus Yao Salivary Gland Protease

I. Detection of fibrinogen hydrolytic activity

Take 10 μL of 2 mg/mL fibrinogen (purchased from Sigma of USA and dissolved in 50 mM Tris-HCl, pH7.4 buffer solution containing 150 mM NaCl ), place it at 37° C. for 24 hr along with the sample. Carry out 12% SDS-PAGE reducing electrophoresis analysis, as shown in FIG. 2. Results show that this enzyme can apparently hydrolyze α-chain of fibrinogen, but its hydrolysis activity for β-chain is low and it cannot hydrolyze γ-chain. Its hydrolysis activity sequence is α>β>γ.

II. Platelet aggregation suppression experiment

Platelet of rich plasma (PRP) aggregation suppression experiment: Dilute healthy person platelet by plasma to 2.5×10⁸ pieces/mL. Take 300 μL of PRP and add suitable amount of sample; keep the mixture warm for 5 min and then add ADP to induce aggregation. Record aggregation of platelet after 5 min.

Calculation of aggregation suppression rate I:

I=B/A×100%

A=maximum aggregation that can be reached by excitant (in transmittance, %);

B=maximum aggregation that can be reached by excitant after use of the sample

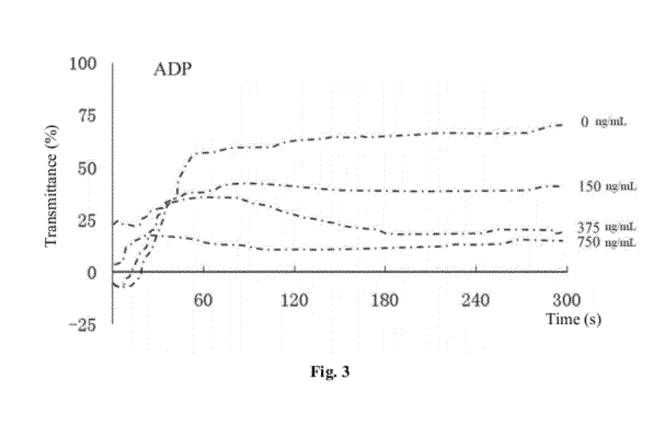

Results show that tabfiblysin can apparently suppress platelet aggregation induced by ADP and the suppression rate increases with increase of inhibitor concentration (FIG. 3).

III. Detection of hemorrhage activity

Dissolve the sample in normal saline so that the concentration is 0.6 mg/mL. Take 30 μL of this solution for hypodermic injection of ICR mice (of body weight of 18-22 g; provided by Kunming Medical College Experimental Animal Center). After 24 hours, peel off mouse skin and observe size of endothelium blood spots. Compare results with a group of mice that adopt normal saline. Results show that tabfiblysin dose as high as 18 μg/mouse will not cause subcutaneous hemorrhage.

Pharmacological experiments of tabanus Yao salivary gland protease show that tabanus Yao salivary gland protease tabfiblysin features apparent effect of hydrolysis of fibrinogen and suppression of platelet aggregation. Tabfiblysin can be used to treat thrombotic diseases.

1. A composition comprising:
a Tabanus Yao protease tabfiblysin;
wherein the Tabanus Yao protease tabfiblysin is an active protein separated from a tabanus Yao salivary gland having a molecular weight of 27145.5 Daltons, and a full-length sequence primary structure of the amino acid sequence shown by SEQ ID NO:2.

2. A composition comprising:
a gene of tabanus Yao protease tabfiblysin, composed of 768 nucleotides, with its 5′ end to 3′ end sequence being the nucleotide sequence shown by SEQ ID NO:1.

3. Application of tabanus Yao protease tabfiblysin, which hydrolyzes fibrinogen and suppress platelet aggregation, without hemorrhage activity, and used to treat thrombotic diseases.
